(19) United States
(12) Patent Application Publication
(10) Pub. No.: US 2004/0010288 A1
(43) Pub. Date: Jan. 15, 2004

(54) Patient intubation positioning device

(76) Inventor: Bassem Ghaly, Chicago, IL (US)

Correspondence Address: FACTOR & PARTNERS, PLC, 305 HOOVER BOULEVARD, SUITE 300, HOLLAND, MI, 49423, US

(21) Appl. No.: 10/373,644

(22) Filed: Feb. 24, 2003

Related U.S. Application Data
(60) Provisional application No. 60/359,173, filed on Feb. 22, 2002.

(51) Int. Cl. A61F 5/00

(52) U.S. Cl. 606/240

(57) ABSTRACT

An intubation positioning device comprising a body, an alignment member and an inflation facilitating means. The body includes a top surface, a bottom surface, and side surfaces. The side surfaces are substantially triangular in configuration, such that the top surface and the bottom surface are substantially oblique to each other. Moreover, upon positioning the bottom surface upon an outer surface, the top surface extends angularly in an upward direction. The top surface, bottom surface and side surfaces cooperate to define an inflatable cavity. The alignment member includes a recess positioned within the top surface of the body. The inflation facilitating means is selectively placeable in fluid communication with the inflatable cavity. Inflation of the inflatable cavity of the body correspondingly facilitates the configuration of the body and the alignment member facilitating the receipt and retention of a portion of the head of a patient in a sniffing position.

### CROSS-REFERENCE TO RELATED APPLICATION

This application claims the priority of U.S. Provisional Patent Application Serial No. 60/359,173 filed Feb. 22, 2002, the entire disclosure of which is incorporated herein by reference.

### BACKGROUND OF THE INVENTION

1. Field of the Invention

The present invention relates in general to the medical field, and, more particularly, to a patient intubation positioning device which facilitates the positioning of a patient.

2. Background Art

The intubation of patients is a common practice in hospitals. While such procedures are rather common, there have been complications. In particular, it is often difficult to properly position the patient. While there is an acceptable range of proper positions, improper placement of the patient can result in trauma during intubation, such as, among others, chipped or broken teeth, lacerations, and/or abrasions.

Certain solutions have been developed to facilitate the positioning of the patient. For example, certain rigid devices for positioning have been proposed. While these devices have assisted, they have certain drawbacks. For example, the devices are generally quite cumbersome and difficult to handle. Moreover, the devices are substantially difficult to store, warehouse and to dispose of upon the completion of the use thereof. Furthermore, it is generally quite difficult to maintain such devices in close proximity to where such devices are utilized due to the cumbersome nature thereof

As a result, it is an object of the present invention to overcome the deficiencies of the prior art.

### SUMMARY OF THE INVENTION

The invention comprises an intubation positioning device comprising a body, an alignment member and an inflation facilitating means. The body includes a top surface, a bottom surface, and side surfaces. The side surfaces are substantially triangular in configuration, such that the top surface and the bottom surface are substantially oblique to each other. Moreover, upon positioning the bottom surface upon an outer surface, the top surface extends angularly in an upward direction. The top surface, bottom surface and side surfaces cooperating to define an inflatable cavity. The alignment member includes a recess positioned within the top surface of the body. The inflation facilitating means selectively placeable in fluid communication with the inflatable cavity, to in turn, facilitate the inflation thereof. Inflation of the inflatable cavity of the body correspondingly facilitates the configuration of the body and the alignment member to, in turn, facilitate the receipt and retention of a portion of the head of a patient in a sniffing position.

In a preferred embodiment, the invention further includes a deflation facilitating means selectively placeable in fluid communication with the inflatable cavity, to, in turn facilitate the deflation thereof. In one embodiment, the deflation facilitating means comprises a release valve.

In another embodiment, the inflation facilitating means further comprises an air connection valve.

Preferably, the cavity of the body is filled with at least one of air, oxygen, foam, etc.

In one embodiment, the alignment member comprises a recess positioned within the top surface of the body.

In yet another embodiment, the top surface of the body is dimensioned so as to be substantially about 2 feet by 1.5 feet.

### BRIEF DESCRIPTION OF THE DRAWINGS

The invention will now be described with reference to the drawings, wherein:

FIG. 1 of the drawings is a top plan view of an embodiment of the present invention;

FIG. 2 of the drawings is a front plan view of an embodiment of the present invention; and

FIG. 3 of the drawings is a side elevational view of an embodiment of the present invention.

### DETAILED DESCRIPTION OF THE INVENTION

While this invention is susceptible of embodiment in many different forms, there is shown in the drawings and described herein in detail several specific embodiments with the understanding that the present disclosure is to be considered as an exemplification of the principles of the invention and is not intended to limit the invention to the embodiments illustrated.

It will be understood that like or analogous elements and/or components, referred to herein, are identified throughout the drawing by like reference characters. In addition, it will be understood that the drawings are merely representations of the invention, and some of the components may have been distorted from actual scale for purposes of pictorial clarity.

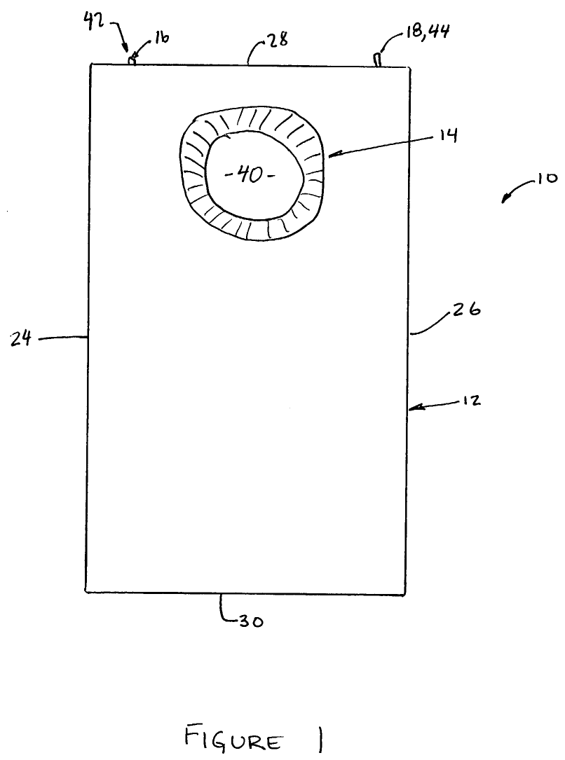

Referring now to the Figures, and in particular to FIG. 1, patient intubation positioning device 10 comprises body 12, alignment member 14, inflation facilitating means 16, and deflation facilitating means 18. Body 12 is shown as comprising top surface 20, bottom surface 22, side surfaces 24, 26, front surface 28, and back surface 30. The various surfaces combine to define an inflatable cavity 29 which, as will be explained, is inflatable by way of inflation facilitating means (i.e., filled with oxygen supplied in hospital, air, foam, etc.). It is contemplated that top surface 20 has approximate dimensions of about 2 feet by 1.5 feet. Front surface 28 is about 8 inches high. While other embodiments are contemplated, as is shown in FIG. 3, side surfaces 24, 26 comprise a triangular configuration wherein the angle of inclination is substantially uniform. Of course other variations are likewise contemplated for use.

Alignment member 14 is shown in FIG. 1 as comprising recess 40 positioned within top surface 20 of body 12. In the contemplated embodiment, recess 40 is configured to receive a portion of the head of a patient, and upon receipt thereof, the head of the patient will be properly positioned for intubation. Generally, the proper position aligns the mouth with the larynx which occurs when the neck is flexed and the head is extended (commonly known as the “sniffing” position). Of course, while the recess is configured for receipt of the head of a patient, additional or different recesses can be incorporated to achieve the desired positioning.

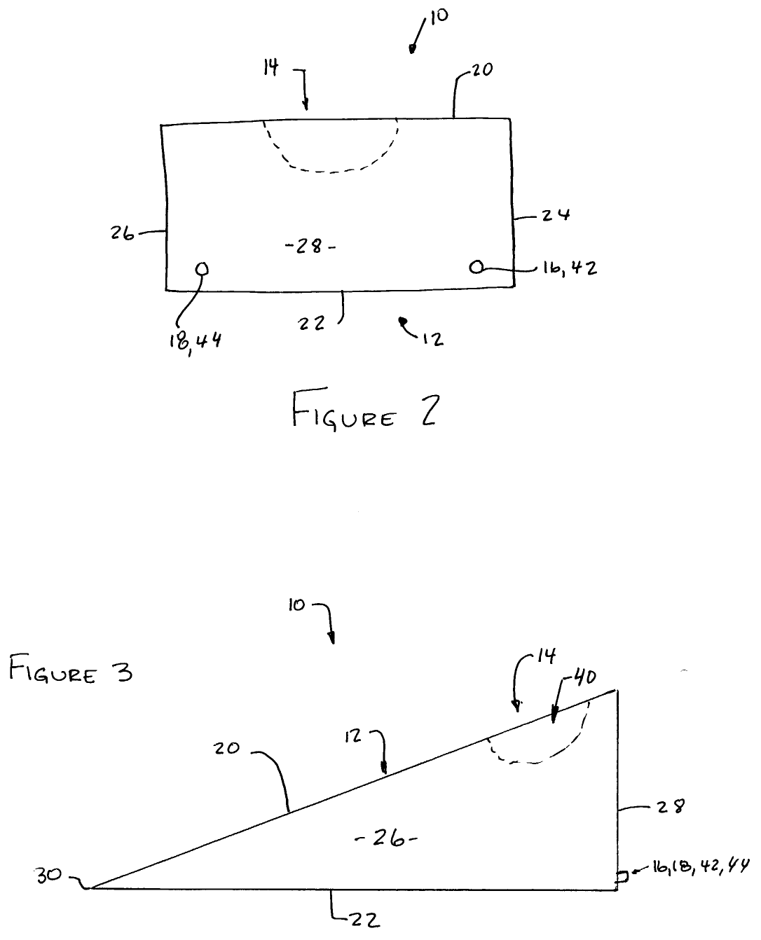

Inflation facilitating means 16 is shown in FIG. 2 as comprising air connection valve 42 which is positioned on front surface 28. The air connection facilitates the inflation of the body to the desired dimensions. As most hospital rooms are equipped with oxygen supplies, the air connection valve may be configured to couple with the existing oxygen hardware in such hospital rooms. Of course, other configurations are likewise contemplated for use. Deflation facilitating means 18 is shown in FIG. 2 as comprising release valve 44 which facilitates the deflation of the body.

In operation, device 10 is first provided and positioned on a bed or other surface. In particular, bottom surface 22 is positioned on the surface. Next, the device is moved so that the alignment member is positioned to correspond to the portion of the patient's body which it is to receive. Of course, the precise positioning is not necessary, however, in the embodiment shown, the device 10 is moved so that the recess is positioned to correspond to the head of the patient.

Next, the device is inflated via inflation facilitating means 16 to the desired pressure. Once completed, the patient can be introduced onto the surface and the proper body member can be positioned within the alignment means. Once the patient is positioned, the doctor can then properly intubate the patient. Once intubated, the device can be removed from the surface, and the patient can be placed on the surface. Advantageously, with the present device, the device can be deflated which makes removal unnecessary.

Of course, it is contemplated that the device may comprise a foam filled body, or a body that is filled with another material. In such an instance, the device can still facilitate proper positioning during intubation, but will require more space for storage and retrieval.

The foregoing description merely explains and illustrates the invention and the invention is not limited thereto except insofar as the appended claims are so limited, as those skilled in the art who have the disclosure before them will be able to make modifications without departing from the scope of the invention.

What is claimed is:

1. An intubation positioning device comprising:
a body including:
a top surface, a bottom surface, and side surfaces, the side surfaces being substantially triangular in configuration, such that the top surface and the bottom surface are substantially oblique to each other, and such that upon positioning the bottom surface upon an outer surface, the top surface extends angularly in an upward direction, the top surface, bottom surface and side surfaces cooperating to define an inflatable cavity;
an alignment member including a recess positioned within the top surface of the body; and
inflation facilitating means selectively placeable in fluid communication with the inflatable cavity, to in turn, facilitate the inflation thereof, whereupon, inflation of the inflatable cavity of the body correspondingly facilitates the configuration of the body and the alignment member to, in turn, facilitate the receipt and retention of a portion of the head of a patient in a sniffing position.

2. The intubation device of claim 1 further including a deflation facilitating means selectively placeable in fluid communication with the inflatable cavity, to, in turn facilitate the deflation thereof.

3. The intubation device of claim 2 wherein the body wherein the deflation facilitating means comprises a release valve.

4. The intubation device of claim 1 wherein the inflation facilitating means further comprises an air connection valve.

5. The intubation device of claim 1 wherein the cavity of the body is filled with at least one of air, oxygen, foam, etc.

6. The intubation device of claim 1 wherein the alignment member comprises a recess positioned within the top surface of the body.

7. The intubation device of claim 1 wherein the top surface of the body is dimensioned so as to be substantially about 2 feet by 1.5 feet.
